(19) United States
(12) Patent Application Publication
(10) Pub. No.: US 2002/0155513 A1
(43) Pub. Date: Oct. 24, 2002

(54) Galectin expression is induced in cirrhotic liver and hepatocellular carcinoma

(76) Inventors: Daniel K. Hsu, Davis, CA (US); Fu-Tong Liu, Davis, CA (US); Christopher Dowling, Freeport, ME (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/975,143

(22) Filed: Oct. 10, 2001

Related U.S. Application Data
(63) Continuation of application No. PCT/US00/08561, filed on Mar. 29, 2000.
(60) Provisional application No. 60/129,111, filed on Apr. 13, 1999.

(51) Int. Cl. C12Q 1/68; G01N 33/574

(52) U.S. Cl. 435/7.23; 435/6

(57) ABSTRACT

The present invention relates to the discovery of a marker for liver disease. Novel diagnostics, prognostics, therapeutics and methods of use of the foregoing for the treatment and prevention of hepatocellular carcinoma are also disclosed.

### CROSS-REFERENCE TO RELATED APPLICATIONS

This application is a continuation of international application number PCT/US00/08561, and claims the benefit of priority of international application number PCT/US00/08561 having international filing date of Mar. 29, 2000, designating the United States of America and published in English, which claims the benefit of priority of U.S. provisional patent application No. 60/129,111, filed Apr. 13, 1999; both of which are hereby expressly incorporated by reference in their entireties.

### FIELD OF THE INVENTION

The present invention relates to the discovery of a marker for liver disease. Novel diagnostics, prognostics, therapeutics and methods of use of the foregoing for the treatment and prevention of hepatocellular carcinoma are also disclosed.

### BACKGROUND OF THE INVENTION

Hepatocellular carcinoma is a major type of cancer causing a quarter of a million deaths worldwide each year (Kew, M. C., Tumors of the liver. In: D. Zakim and T. D. Boyer (eds.), Hepatology. A Textbook of Liver Disease, pp. 1206-1240, W. B. Saunders, Philadelphia (1990)). While various factors have been identified as causes for HCC, the major established factors are infections of hepatitis viruses B (HBV) and C (HCV). Various potential mechanisms exist whereby infection by HBV can result in HCC. These include both chronic liver injury caused by cytotoxic T cell responses to infected hepatocytes and intracellular occlusion resulting from expression of viral protein (Chisari, F. V., Analysis of hepadnavirus gene expression, biology, and pathogenesis in the transgenic mouse. In: Current Topics in Microbiology and Immunology, pp. 85-99, Springer-Verlag, Berlin (1991)).

Deregulated expression of a number of proteins such as tumor suppressor genes and altered cellular activity of other cellular genes have been associated with HCC. (Robinson, W. S., J. Gastroenterol. Hepatol, 8:95 (1993)). Transactivation by HBV-X protein is also considered to significantly contribute to cell transformation. This protein has been shown to transactivate several viral and cellular genes such as c-jun, c-myc, β-interferon and class I histocompatibility complex. (Robinson, W. S., J. Gastroenterol. Hepatol, 8:95 (1993)). The transcription factors CREB and ATF2 and various cis-acting DNA elements that regulate cellular genes, such as 6B, AP1 and AP2, are also affected by HBV-X. (Robinson, W. S., J. Gastroenterol. Hepatol, 8:95 (1993)). Although significant strides have been made toward understanding the onset of HCC, the need for sensitive diagnostics, prognostics, and therapeutics for the treatment and prevention of HCC, is manifest.

### BRIEF SUMMARY OF THE INVENTION

Aspects of the present invention relate to the discovery that hepatocytes in liver biopsies from subjects with hepatocellular carcinoma (HCC) have significant levels of galectin-3 (76% immunohistochemically positive) while galectin-3 expression is low or absent in normal hepatocytes. Notably, the correlation of galectin-3 expression and HCC is independent of whether the subject had prior hepatitis B virus infection (14 of 18 HCC cases from HBV+ patients, and 5 of 7 cases from HBV− patients demonstrated positive galectin-3 immunohistochemistry). This indicates that galectin-3 expression may be used to detect HCC resulting from a variety of factors. However, co-transfection studies using a galectin-3 promoter construct and an HBV-X protein (HBV-X) expression vector demonstrated that galectin-3 expression can occur through transactivation of the galectin-3 promoter by HBV-X. Additionally, aspects of the present invention relate to the discovery that galectin-3 is abundantly expressed in cirrhotic liver in a peripheral distribution within regenerating nodules. The galectin-3 expression in rapidly proliferating hepatocytes in cirrhotic liver can be used to identify an early neoplastic event.

### BRIEF DESCRIPTION OF THE DRAWINGS

FIG. 1 shows the results of two chloramphenicol acetyl transferase assays in which the transactivation of the galectin-3 promoter by co-transfection with an HBV-X protein expression vector is demonstrated.

FIG. 2 shows the cDNA sequence of human galectin-3.

### DETAILED DESCRIPTION OF THE INVENTION

In the present invention, several embodiments relate to the discovery that hepatocytes in liver biopsies from subjects with hepatocellular carcinoma (HCC) have significant levels of galectin-3 (76% immunohistochemically positive); whereas, galectin-3 expression is low or absent in normal hepatocytes. In addition, galectin-3 expression in HCC is independent of whether the subject had prior hepatitis B virus infection (14 of 18 HCC cases from HBV+ patients, and 5 of 7 cases from HBV− patients demonstrated positive galectin-3 immunohistochemistry). Furthermore, co-transfection of a galectin-3 promoter construct and an HBV-X protein (HBV-X) expression vector induces galectin-3 expression, through transactivation of the lectin promoter by HBV-X. Additionally, galectin-3 expression in regenerating nodules is also associated with cirrhosis of the liver. Herein we provide novel diagnostics and prognostics for determining whether a subject is suffering from HCC or cirrhosis of the liver or is likely to develop HCC or cirrhosis of the liver. Therapeutic agents or vaccine components for the treatment and/or prevention of HCC or liver cirrhosis are also provided.

Galectins are a family of proteins characterized by sequence homologies in a domain which exhibits galactose-specific carbohydrate binding activity. There are presently 12 characterized eukaryotic members, and currently a total of two dozen members have been predicted by homology from DNA sequences present in databases. Several dozen human members can be anticipated from projections based on the number of similar members present in the completely sequenced Caenorhabditis elegans genome. Galectins do not contain traditional sequences that specify membrane translocation, but are both secreted and located intracellularly. Of the characterized galectins, galectins-1 and 3 are the most extensively studied (Barondes, et al., J. Biol. Chem., 269:20807 (1994); Kasai, K. and Hirabayashi, J., J. Biochem. (Tokyo), 119:1 (1996)).

Galectin-3, previously designated as εBP, CBP35, Mac-2, L-29 and L-34, has been associated with assorted processes such as cell growth, tumor transformation, and metastasis. (Moutsatsos, et al., Proc. Natl. Acad. Sci. USA, 84:6452 (1987), (Raz, et al., Cancer Res., 46:3667 (1986), (Raz, et al., Int. J. Cancer, 46:871 (1990), and (Hsu, et al., Am. J. Pathol., 148:1661 (1996)). This lectin of approximately 30,000 Da is composed of two domains: a carboxyl-terminal domain that contains the carbohydrate-binding region which binds saccarides with terminal galactose residues and an amino-terminal domain consisting primarily of tandem repeats of nine amino acids (Liu, et al., Biochemistry, 35:6073 (1996)). Galectin-3 is expressed in various tissues and organs and is present in dendritic cells and a variety of normal cells of epithelial origin but is undetectable in normal lymphocytes and hepatic cells.

One aspect of the present invention relates to methods of determining whether an individual is suffering from HCC or cirrhosis of the liver, or is likely to suffer from HCC or cirrhosis of the liver, comprising determining whether the level of galectin-3 expression in the subject's hepatocytes is above normal. Example 1 describes the immunohistochemical characterization of galectin-3 expression in samples from normal liver, subjects with cirrhosis of the liver, and subjects with HCC. Pathological specimens were obtained from archives at the Medical Center of the University of California, San Diego, or the Veterans General Hospital, Taichung, Taiwan. Cases studied included normal liver (six cases), cirrhotic liver (eight cases), hepatocellular carcinoma (25 cases), fibrolamellar tumor (two cases) and hepatoblastoma (one case).

### EXAMPLE 1

The expression of galectin-3 in normal human liver and cirrhotic human liver biopsy tissue was analyzed by immunohistochemistry. These normal liver biopsies were obtained from normal subjects or regions in HCC patients unaffected by tumor. Tissues were fixed in either paraformaldehyde (for samples probed with rabbit antibody) or B5 (for samples probed with monoclonal antibody) and embedded in paraffin. Tissue sections were processed for immunohistochemical detection of galectin-3, as described previously (Konstantinov, et al., Am. J. Pathol., 148:25 (1996)). In brief, primary antibodies used were affinity-purified rabbit anti-galectin-3 antibody or mouse monoclonal anti-galectin antibody A3A12 (Liu, et al., Biochemistry, 35:6073 (1996)), both at 10 μg/ml. Secondary antibodies were goat anti-rabbit peroxidase and goat anti-mouse peroxidase conjugates, respectively. Antibody controls used were normal rabbit immunoglobulin and isotype matched irrelevant monoclonal antibodies, and did not result in any staining. Chromogenic substrates were 3,3′-diaminobenzidine or 3-amino-9-ethylcarbazole.

A total of four normal samples were probed with affinity purified rabbit polyclonal anti-galectin-3. Positive reactivities were demonstrated by brown precipitates (peroxidase-diaminobenzidine) and counterstaining was done with hematoxylin. Prominent staining was noted in epithelial cells lining the bile ducts and cells with the appearance and distribution of Kuppfer Cells. In contrast, hepatocytes did not show any significant level of staining. Nor did connective tissue show any staining. The negative controls for the experiment used non-immune rabbit gamma globulin instead of the specific primary antibodies and consistently showed only background staining. Two normal biopsies were also reacted with monoclonal antibodies and similar reactivities to galectin-3 as that obtained with the polyclonal antibody were obtained. The observed pattern of galectin-3 expression in normal human liver is consistent with that reported for mouse liver. (Flotte, et al., Am. J. Pathol., 111:112 (1983)).

The most prominent feature observed in the eight biopsies from subjects having cirrhosis of the liver was the strong staining of focal regenerating nodules. Variable degrees of staining intensity were seen in different foci of regenerating liver. Preferential circumferential expression of galectin-3 in the foci was also observed, suggesting differential regulation. Islands of positively stained cells, and general distribution of other positively staining cells were also observed throughout nodules. In all samples of cirrhotic liver, both cytoplasmic and nuclear expression of galectin-3 were observed. Other regions of hepatic tissue do not display this staining pattern. The ductal epithelial cells, dendritic cells, and Kuppfer cells were also positively stained in the tissue specimens from cirrhotic liver. In the discussion that follows, we describe an experiment in which we discovered that galectin-3 is expressed in human hepatocellular carcinoma.

The pattern of galectin-3 expression in human hepatocellular carcinoma was visualized in much the same manner as described above for normal and cirrhotic liver. The pattern of galectin-3 expression was determined by using an affinity purified rabbit anti-galectin-3 antibody as the primary antibody and positive reactivities were demonstrated by reddish-brown precipitates (peroxidase-aminoethylcarbazole). In addition, a mouse monoclonal anti-galectin-3 antibody was used and positive reactivities were observed by brown precipitates (peroxidase-diaminobenzidine). In both sets of experiments, counterstaining was done with hematoxylin.

The immunohistochemical staining of tumor cells revealed varying degrees of intensity and distribution. Based on the intensity and distribution of positive staining regions, scores from 1 to 4 were assigned: One+ (scattered positive tumor cells); 2+ (diffuse positive or focally positive); 3+ (large areas of positives, diffuse and focal); and 4+ (mostly positive and strongly positive). In most of the HCC tissues (76%) positive galectin-3 immunoreactivities of scoring intensity 1 and greater were observed in the neoplastic cells. (See Table I). In each HCC tissue sample, localization of galectin-3 was predominantly cytoplasmic but nuclear staining was invariably noted in many cells, and was observed in all samples positive for galectin-3 expression. The neoplastic cells were not uniformly positive and tissues from different cases were variable in terms of the fraction of the tumor cells that were positively stained. In some specimens, however, the neoplastic cells were not stained despite positive staining of the bile ducts and Kuppfer cells. Specificity of the rabbit anti-galectin-3 antibody was verified by immunohistochemistry using a mouse monoclonal antibody. Two HCC biopsies were stained with both polyclonal and monoclonal antibodies and similar staining patterns in tissues expressing the lectin were observed. For most cases of hepatocellular carcinoma, HBV serology of the patients was available, and are included in Table I. Positive HBV infection status is defined by presence of serum HBV surface antigen or antibodies to HBV surface antigen. Positive and negative sera status are indicated as ‘+’ and ‘−’, respectively, and no signage when no information was available (Table I). Of the 25 HCC biopsies studied, 18 were from documented HBV seropositive patients. Seventy-eight percent of the 18 biopsies showed positive immunohistochemical reactivities for galectin-3. Seven cases of HCC occurred in HBV sero negative patients, five of which were positively stained for galectin-3. Thus, galectin-3 expression in hepatocytes is a general marker for HCC, and is not limited to HCC resulting from HBV infection. The HCC samples studied for galectin-3 expression and the serological status for HBV antigens are summarized in Table I.

TABLE I
Galectin-3 Immunohistochemical Reactivities in HCC Tissue

| Patient | ImmHistChem | | | | | |
|---|---|---|---|---|---|---|
| # | Grading | HBsAg | HBeAg | α-HBs | α-HBe | α-HBc |
| TW-1 | 1+ | + | + | − | − | + |
| TW-2 | 3+ | − | − | + | | |
| TW-3 | 1+ | − | − | + | | + |
| TW-4 | 1+ | + | − | − | + | + |
| TW-5 | 1+ | + | | − | | |
| TW-6 | 3+ | + | + | − | − | + |
| TW-7 | 1+ | + | − | | | |
| TW-8 | 3+ | − | | + | | |
| TW-9 | 4+ | + | | − | − | + |
| TW-10 | 1+ | − | − | + | + | − |
| TW-11 | 1+ | − | − | + | + | + |
| TW-12 | 1+ | + | | | | |
| TW-13 | 1+ | + | + | − | − | + |
| TW-14 | 1+ | − | − | + | − | + |
| TW-15 | 0 | + | | − | | |
| TW-16 | 0 | + | − | | + | + |
| TW-17 | 0 | + | − | − | + | + |
| TW-18 | 0 | + | − | − | + | + |
| TW-19 | 3+ | − | | | | |
| TW-20 | 0 | | | | | |
| UC-1 | 3+ | − | | | | |
| UC-2 | 3+ | | | | | |
| UC-3 | 3+ | | | | | |
| UC-4 | 0 | − | | | | |
| UC-5 | 2+ | − | | | | |
| UC-6¹ | 3+ | | | | | |
| UC-7¹ | 3+ | − | | | | |
| UC-8² | 0 | − | | | | |

Samples classified as HBV positive as shown by serology
are indicated in bold type.
¹Fibrolamellar tumor
²Hepatoblastoma

Summary of HCC Statistics

| | Galectin-3 Positive | Galectin-3 Negative |
|---|---|---|
| HBV+ | 14 | 4 |
| HBV−/undetermined | 5 | 2 |

Abbreviations: HBsAg, HBV surface antigen; HBeAg, HBV envelope antigen; α-HBs, antibodies to HBV surface antigen; α-HBe, antibodies to HBV envelope antigen; α-HBec, antibodies to HBV core antigen.

Another aspect of the present invention relates to methods of determining the pattern of galectin-3 expression in hepatocellular carcinoma cell lines, comprising identifying whether the level of galectin-3 expression in the cells is above normal. Example 2 describes the characterization of galectin-3 expression in several hepatocellular carcinoma cell lines.

### EXAMPLE 2

The association of galectin-3 expression with HCC was also confirmed in established HCC cell lines by immunoblot. As a control, organ extracts from mouse liver, as well as, thymus and spleen were analyzed. The human hepatoma cell lines Hep 3B (HBsAg⁺), Hep G2 (Aden, et al., Nature, 282:615 (1979)), HuH-7 (Nakabayashi, et al., Cancer Res., 42:3858 (1982)) and PLC/PRF/5 (HBsAg⁺) (Alexander, et al., S. Afr. J. Med. Sci., 41:89 (1976)) were kindly provided by Dr. F. Chisari, The Scripps Research Institute, La Jolla, Calif. The M12.4.5 line was a generous gift of Dr. Richard Asofsky, National Institutes of Health, Bethesda, Md. Cells were cultured in Dulbecco's Modified Eagle's Medium supplemented with 10% fetal bovine serum and 2 mM glutamine.

Galectin-3 expression in mouse tissues and hepatoma cell lines was detected by immunoblotting, as described previously (Liu, et al., Am. J. Pathol., 147:1016 (1995)). Briefly, cell lysates were adsorbed with lactosyl-Sepharose 4B and the bound proteins were eluted and electrophoresed in 12.5% polyacrylamide gel. The separated proteins were transferred to Immobilon P membranes and galectin-3 was detected by a chemiluminescent system using a rabbit anti-galectin-3 antibody and alkaline phosphatase-conjugated goat anti-rabbit antibody. Each lane represents the galectin-3 present in 100 μg protein from tissue extracts and 500 μg protein from each cell lysate. Protein markers are shown on the left in kDa.

The control immunoblot showed that galectin-3 was expressed in spleen and thymus extracts but not liver. The immunoblot of the HCC cell lines showed that galectin-3 was expressed in all four HCC cell lines tested. The identity of the protein was also verified by its lectin activity, as demonstrated by binding to lactosyl-Sepharose 4B, co-migration with the authentic galectin-3 in electrophoresis, and reactivity with specific rabbit antibodies in immunoblot. Two HCC cell lines used, Hep 3B and PLC/PRF/5 are known to contain integrated HBV sequences (Aden, et al., Nature, 282:615 (1979); MacNab, et al., British J. Cancer, 34:509 (1976)). However, there is no evidence that the other cell lines, Hep G2 and HuH-7 are infected with HBV (Aden, et al., Nature, 282:615 (1979); Nakabayashi, et al., Cancer Res., 42:3858 (1982)). Galectin-3 expression appeared to be generally associated with HCC but is not limited to HCC caused by HBV.

While galectin-3 expression is not limited to HCC resulting from HBV, HBV-X protein induces galectin-3 expression, as described in the example below.

### EXAMPLE 3

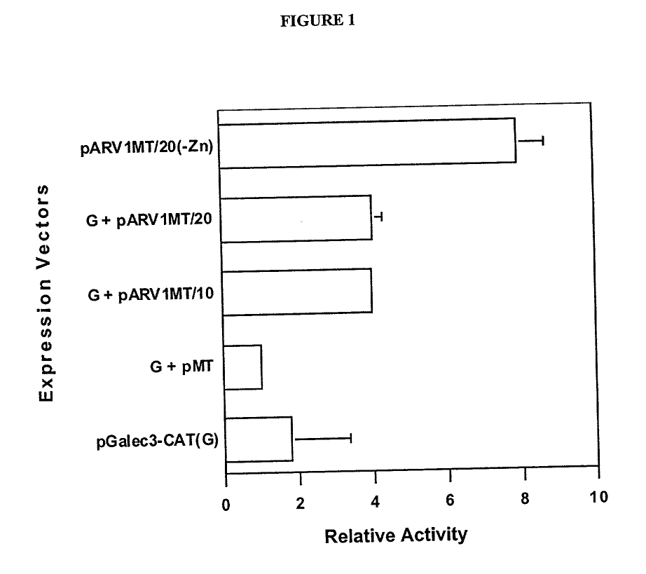

The expression of galectin-3 in HBV seropositive individuals with HCC prompted us to test whether the galectin-3 promoter can be activated by HBV transactivating proteins. (See FIG. 1). Accordingly, the vector pGalec3-CAT, which contains the murine galectin-3 promoter coupled to a chloramphenicol acetyl transferase (CAT) reporter gene, was used to co-transfect M12.4.5 cells with a vector expressing the HBV transactivating protein HBV.X. Construction of the vector pGalec3-CAT consisting of the murine galectin-3 promoter upstream of a bacterial chloramphenicol acetyl transferase (CAT) gene was accomplished, as described by Hsu, et al., Am. J. Pathol., 148:1661 (1996). Briefly, a 2 kb region upstream from the transcription initiation site was inserted into a vector housing a CAT gene. The HBV-X expression vector pARV1MT and parent pMT (McLachlan, et al., J. Virol., 61:683 (1987)), used for co-transfections, were generous gifts of Dr. Alan McLachlan, The Scripps Research Institute, and contained the murine metallothionein-I gene promoter sensitive to and inducible with metal ions. Co-transfections of pGalec3-CAT and pARV1MT or pMT into murine M12.4.5 cells were accomplished by electroporation, as described by Hsu, et al., Am. J. Pathol., 148:1661 (1996). Induction of the metallothionein promoter was initiated in some cultures twenty four hr after electroporation by addition of ZnCl₂ to 10 μM. CAT activities of transfectants were assayed 48 hr after transfection using the phase transfer method with [dichloroacetyl-1,2- ¹⁴C]-chloramphenicol (New England Nuclear, Boston, Mass.) and n-butyl coenzyme A (Sigma, St. Louis, Mo.). Corrections were made for basal activity by subtracting the activity of the parental pCAT-basic vector from all other measurements.

The CAT activity shown in FIG. 1 is a measure of the activity of the galectin-3 promoter in the pGalec3-CAT vector in M12.4.5 host cells. Cotransfections were performed with the HBV-X expression vector pARV1MT or control pMT. Cotransfections performed with 10 μg or 20 μg pARV1MT in the presence of zinc are indicated by pARV1MT/10 and pARV1MT/20, respectively, and pARV1MT/20(-Zn) when performed with 20 μg vector in the absence of zinc. Significant upregulation of the galectin-3 promoter is observed when co-transfected with pARV1MT, but not with pMT. All values were corrected for basal CAT activity reflective of transfection with the promoter-less CAT expression vector, and are expressed relative to pGalec3-CAT alone (unity). Data are means of duplicate transfections shown with error bars indicating SD and are representative of 2 experiments.

As shown in FIG. 1, co-transfection of the vector pGalec3-CAT and the vector expressing HBV-X protein resulted in upregulation of the galectin-3 promoter as evident from increased CAT activity. The vector expressing HBV-X protein contains a metallothionein gene promoter and is sensitive to and inducible by metal ions. In the presence of Zn²⁺, however, suppression of CAT activity was observed, indicating that activation of the murine metallothionein-I promoter driving expression of the HBV-X protein had already been achieved.

The results presented above show that many normal hepatocytes, which do not express galectin-3, are converted to galectin-3 expressing cells upon neoplastic transformation. Of the HCC biopsies evaluated, 19 of 25 (76%) were immunohistochemically positive for galectin-3. While not all neoplastic cells in HCC express this lectin and the cases of HCC studied fall into a spectrum in terms of the proportion of tumor cells that are positively stained for galectin-3; galectin-3 expression is associated with HCC and cirrhosis of the liver. The presence of galectin-3 in HCC cell lines indicates that upregulation of this lectin occurs in transformed hepatocytes.

Analysis of galectin-3 expression in HCC from patients with or without serological evidence of HBV infection revealed that galectin-3 expression in HCC is not strictly correlated with HBV infection (positive galectin-3 expression in 78% of HBV+ patients and 71% of HBV− patients). In addition, since at least one of the cell lines shown to express galectin-3 does not appear to exhibit evidence of HBV infection, the data suggest that factors other than HBV infection can result in galectin-3 expression. Additional evidence indicating non-viral upregulation of galectin-3 expression in hepatic tissue exists in the observation that fibrolamellar tumors also express galectin-3. These tumors generally occur in young patients, are not associated with chronic infection with HBV, and do not usually appear in cirrhotic liver (Berman, et al., Cancer, 46:1448 (1980); Craig, et al., Cancer, 46:372 (1980)).

The following discussion is offered only to provide possible explanations for the relationship of expression of galectin-3 to neoplasia. These explanations are not intended to limit any aspect of the present invention nor are they intended to be used to construe, interpret, or restrict the scope of protection provided by the claims and/or the scope of equivalent compositions and methods.

Results from our co-transfection experiments show that the upregulation of galectin-3 in HBV infected and transformed cells can result from transactivation of the galectin-3 promoter by the HBV-X protein. No direct sequence-specific interaction of this protein occurs with DNA (Wu, et al., Cell, 63:687 (1990)). The effects of HBV-X, however, can be mediated by interactions with transcription factors CREB and ATF2 (Maguire, et al., Science, 252:842 (1991)). In addition, HBV-X can transactivate DNA elements regulating viral and cellular genes such as the 6B sequence bound by the transcription factor NF-6B (Twu, et al., Proc. Natl. Acad. Sci. USA, 86:5168 (1989), the c-myc gene promoter (Koike, et al., Molec. Biol. Med., 6:151 (1989) and binding sites for AP1 and AP2 transcription factors (Seto, et al., Nature, 344:72 (1990)). Indeed, several potential DNA sites for interaction with NF-6B, AP1 and AP2 were identified within 1 kb upstream of the transcription initiation site. The transactivation of the galectin-3 gene by HBV-X is, therefore, consistent with our data demonstrating upregulation of this promoter by HTLV-I Tax (Hsu, et al., Am. J. Pathol., 148:1661 (1996), a potent viral transactivator through the CREB/ATF transcriptional factor pathway. Furthermore, upregulated expression of galectin-3 was also demonstrated in 3T3 cells following transformation with the Kirsten murine sarcoma virus (Crittenden, et al., Mol. Cell Biol., 4:1252 (1984)). Additionally, analysis of fibroblasts from mouse and chicken for galectin-3 expression showed that infection with viruses can result in upregulated expression of the lectin (Crittenden, et al., Mol. Cell Biol., 4:1252 (1984)). In sum, we believe that galectin-3 expression may be affected by many viruses.

The finding that regenerating nodules in cirrhotic liver express galectin-3 is also worthy of comment. Since these hepatocytes are undergoing rapid proliferation, we believe that this proliferative cell status is linked to galectin-3 expression. Previously, others reported that elevated levels of this lectin are found in proliferating fibroblasts (Moutsatsos, et al., Proc. Natl. Acad. Sci. USA, 84:6452 (1987)). An alternative but not mutually exclusive explanation may be the following. Because oval cells expressing markers for both bile duct epithelia and hepatocytes (Gerber, et al., Am. J. Pathol., 110:70 (1983)) are hypothesized to function as hepatic stem cells (Fausto, N. and Thompson, N. L., Purification and culture of oval cells from rat liver. In: Pretlow II, T. G. and T. P. Pretlow (eds.), Cell Separation: Methods and Selected Applications, pp. 45-96, Academic Press, Orlando (1986)) and since galectin-3 is expressed in cells of ductal origin, presence of this protein in these nodules may be residual lectin resulting from the production of hepatocytes from these stem cells. In either event, we believe that regulation of galectin-3 expression during differentiation of hepatocytes relates to the fate of the cells, and deregulation, as a result of viral influences, interferes with this process. Further, we contemplate that cells positively stained for galectin-3 are in the process of becoming cancer cells and that expression of galectin-3 represents an early event leading to tumor transformation. Alternatively, expression of galectin-3 in cirrhotic cells may indicate conversion to normalcy.

In the following discussion, we provide several diagnostic and prognostic tools for use with embodiments of the present invention.

### Diagnostic Tools

While normal hepatocytes do not express galectin-3, expression of this lectin is associated with human hepatocellular carcinoma (HCC). Lectin expression in HCC is positively influenced by hepatitus B virus (HBV) infection through a mechanism that includes transactivation of the galectin-3 gene promoter. Other areas in the liver found to express galectin-3 are focal regenerating nodules of cirrhotic tissue. We contemplate that deregulated expression of galectin-3 results in transformation, conversion of tumors to increased invasiveness, and confers preferential survival of tumors. Accordingly, several diagnostic and prognostic tools which detect expression of galectins in the liver may be used to determine whether an individual is suffering from HCC or cirrhosis of the liver or is likely to suffer from HCC or cirrhosis of the liver in the future.

Generally, the diagnostics and methods of use thereof can be classified according to whether the diagnostic detects the expression of galectin-3 protein in a biological sample (e.g., a sample having hepatocytes) or the expression of galectin-3 RNA in a biological sample. Accordingly, the level of expression of galectin-3 protein or RNA in a biological sample indicates a predilection to liver disease (e.g., cirrhosis or HCC). Once a biological sample from a subject in need of testing is obtained, many different techniques can be used to detect the level of galectin-3 protein and/or RNA expression including, but not limited to, antibody-based detection techniques, bacteriophage display techniques, lectin-binding techniques, hybridization techniques, and enzymatic digestion (e.g., RNAse protection) techniques. Additionally, we contemplate the use of physical detection methods including, but not limited to, absorption, emission, or resonance spectra, and the identification of galectins by nucleic acid or protein fragmentation patterns (e.g., enzymatic or chemical cleavage patterns). Some of these techniques involve disposing the proteins and/or nucleic acids present in the biological sample on a support, and contacting the support with detection components such as antibodies to galectin-3 or nucleic acid probes complementary to galectin-3 mRNA. Desirably, the levels of expression of galectin-3 protein and/or RNA from diseased and normal individuals is compared to the level detected in the subject tested.

Additionally, we contemplate the preparation of diagnostic kits comprising detection components such as antibodies specific for the galectin -3 protein or nucleic acid probes for detecting RNA encoding galectin-3. The detection component will typically be supplied in combination with one or more of the following reagents. A support capable of absorbing or otherwise binding RNA or protein will often be supplied. Available supportS for this purpose include, but are not limited to, membranes of nitrocellulose, nylon or derivatized nylon that may be characterized by bearing an array of positively charged substituents. One or more enzymes, such as Reverse Transcriptase and/or Taq polymerase, may be furnished in the kit, as may dNTPs, buffers, or non-human polynucleotides like calf-thymus or salmon-sperm DNA. Results from the kit assays can be interpreted by a healthcare provider or a diagnostic laboratory. Alternatively, diagnostic kits are manufactured and sold to private individuals for self-diagnosis.

In many aspects of the present invention, galectins or RNA encoding galectins obtained from a biological sample are disposed on a support so that the level of galectin expression can be rapidly determined. The term “matrix” or “support” refers to a carrier, a resin or any macromolecular structure used to attach, immobilize, or dispose thereon a biomolecule such as RNA or protein. Solid supports include, but are not limited to, the walls of wells of a reaction tray, test tubes, polystyrene beads, magnetic beads, nitrocellulose strips, membranes, microparticles such as latex particles, sheep (or other animal) red blood cells, duracytes® and others. Additionally, organic carriers including proteins and oligo/polysaccarides (e.g. cellulose, starch, glycogen, chitosane or aminated sepharose) and inorganic carriers such as silicon oxide material (e.g. silica gel, zeolite, diatomaceous earth or aminated glass) are contemplated. Furthermore, in some embodiments, a liposome or lipid bilayer (natural or synthetic) is contemplated as a support. Desirable supports also include polyacrylamide gels, agarose gels, composite gels, and other gel matrices, papers, chips, membranes, chromatography matrices, as used in thin layer chromatography, and resins or beads, as used in affinity chromatography.

The support may have a hydrophobic surface which interacts with a portion of the biomolecule by hydrophobic non-covalent interaction. For example, the hydrophobic surface of the support may be a polymer such as plastic or any other polymer in which hydrophobic groups have been linked such as polystyrene, polyethylene or polyvinyl. The support may also have a charged surface which interacts with the biomolecule such as, a charged nitrocellulose or nylon membrane. The supports may have other reactive groups which can be chemically activated so as to attach a biomolecule. For example, cyanogen bromide activated matrices, epoxy activated matrices, thio and thiopropyl gels, nitrophenyl chloroformate and N-hydroxy succinimide chlorformate linkages, and oxirane acrylic supports are common in the art. (Sigma)

Any biomolecule which interacts with a galectin or a nucleic acid sequence complementary to a nucleic acid encoding a galectin (e.g., antibody, phage, 3-galactoside sugar, other galectin ligand, or DNA or RNA), may be attached in overlapping areas or at random locations on the solid support and can be used to probe for the presence of a galectin or RNA encoding a galectin in a biological sample. Additionally, biomolecules which interact with galectins or nucleic acid sequences encoding galectins may be attached in an ordered array wherein each polynucleotide is attached to a distinct region of the solid support which does not overlap with the attachment site of any other biomolecule. Preferably, such an ordered array of biomlecules is designed to be “addressable” where the distinct locations are recorded and can be accessed as part of an assay procedure. Addressable biomolecule arrays typically comprise a plurality of different biomolecule probes that are coupled to a surface of a substrate in different known locations. The knowledge of the precise location of each biomolecule location makes these “addressable” arrays particularly useful in binding assays.

Any addressable array technology known in the art can be employed with this aspect of the invention. One particular embodiment of polynucleotide arrays is known as the Genechips™, and has been generally described in U.S. Pat. No. 5,143,854; PCT publications WO 90/15070 and 92/10092. These arrays may generally be produced using mechanical synthesis methods or light directed synthesis methods, which incorporate a combination of photolithographic methods and solid phase oligonucleotide synthesis (Fodor et al., Science, 251:767-777, 1991). The immobilization of arrays of oligonucleotides on solid supports has been rendered possible by the development of a technology generally identified as “Very Large Scale Immobilized Polymer Synthesis” (VLSIPS™) in which, typically, probes are immobilized in a high density array on a solid surface of a chip. Examples of VLSIPS™ technologies are provided in U.S. Pat. Nos. 5,143,854 and 5,412,087 and in PCT Publications WO 90/15070, WO 92/10092 and WO 95/11995, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and sequence information. Examples of such presentation strategies are disclosed in PCT Publications WO 94/12305, WO 94/11530, WO 97/29212 and WO 97/31256.

In the following discussion, we provide methods and compositions which are used to detect the presence of galectin-3 protein in a biological sample so that the predisposition to HCC can be determined.

Detection of galectin-3 expression using protein-based diagnostics

By one approach galectin-3 expression is determined by using protein-based diagnostics. The presence and amount of galectin-3 protein in a biological sample can be detected by screening for the presence of the protein using conventional assays. Antibodies (monoclonal or polyclonal) immunoreactive with galectin-3 protein can be used to screen biological samples such as serum, urine, ascites, cerebrospinal fluids or any other body fluid. Preferably, liver cells, extracts of liver cells, or liver biopsy tissue, as described above, are screened for the presence and amount of galectin-3. Portions, fragments, or antibody derivatives are contemplated for use with several embodiments of the present invention. Further, phage display or galactin-3 binding assays can be used to detect the presence and amount of galectin-3 in a biological sample. Such immunological assays can be done in many convenient formats and are known to those of skill in the art.

In some embodiments, antibodies reactive to galectin-3 are used to immunoprecipitate galectin-3 protein from a liver cell homogenate. In others, Western or Immunoblots of proteins isolated from liver cells are performed using anti-galectin-3 antibodies. In another preferred embodiment, antibodies will detect galectin-3 in paraffin or frozen sections of liver tissue, using immunocytochemical techniques. Preferred embodiments relating to methods for detecting the presence and amount of galectin-3 protein also include enzyme-linked immunosorbant assays (ELISA), radioimmunoassays (RIA), immunoradiometric assays (IRMA), fluorescent activated cell sorting (FACS), and immunoenzymatic assays (IEMA), including sandwich assays using monoclonal and/or polyclonal antibodies. Exemplary sandwich assays are described by David et al., in U.S. Pat. Nos. 4,376,110 and 4,486,530.

The presence of galectin-3 in a sample may also be determined using affinity chromatography columns having a 3-galactoside bound thereto. The proteins present in a biological sample are applied to a support having a 3-galactoside sugar (e.g., a lactosyl-SEPHAROSE resin) under conditions which allow galectin-3 to bind to the support. The presence and amount of galectin-3 can be determined directly using antibodies or phage display or the bound galectin-3 can be eluted by using a 3-galctoside sugar (e.g., 0.1M lactose). Many more methods for accessing the ability galectin-3 protein to bind a 3-galactoside sugar are known to the art.

In order to provide a basis for diagnosis, normal or standard values for galectin-3 expression of a subject is preferably established. This can be accomplished in many ways and one approach is to contact body fluids, cell extracts, or biopsy tissue from normal subjects with antibody to galectin-3 under conditions suitable for complex formation. The amount of antibody bound to normal fluids, cells, and tissues (e.g., liver cells and liver tissue) is quantified and recorded using conventional immunological techniques. This measurement can serve as a baseline to which biological samples from subjects having HCC or cirrhosis of the liver or likely to develop HCC or cirrhosis of the liver can be compared. Additionally, databases having measurements of galectin-3 expression of several afflicted individuals (similar to that shown in Table 1) are valuable standards by which the progression of liver disease, based on galectin-3 expression, can be monitored. In this manner, deviation between the standard and the subject values establishes the presence and severity of disease state.

In the following discussion, we provide methods and compositions which are used to detect the presence of RNA encoding galectin-3 in a biological sample so that the presence of or predisposition to HCC and cirrhosis can be determined. Detection of galectin-3 expression using RNA-detection diagnostics

A wide variety of labels and conjugation techniques are known by those skilled in the art and can be used in various nucleic acid assays. There are several ways to produce labeled nucleic acids for hybridization or PCR (Polymerase Chain Reaction) including, but not limited to, oligolabeling, nick translation, end-labeling, or PCR amplification using a labeled nucleotide. Alternatively, a desired galectin sequence, or any portion of it, may be cloned into a vector for the production of an mRNA probe. Such vectors are known in the art, are commercially available, and may be used to synthesize RNA probes in vitro by addition of an appropriate RNA polymerase such as T7, T3 or SP6 and labeled nucleotides. A number of companies such as Pharmacia Biotech (Piscataway N.J.), Promega (Madison Wis.), and U.S. Biochemical Corp (Cleveland Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like.

For diagnostic and prognostic purposes, nucleic acid probes having a sequence complementary to a nucleic acid encoding a galectin may be used to detect and quantitate gene expression of the galectin in biological samples including, but not limited to biopsied tissues or any of the biological samples discussed above. Preferably, nucleic acid probes which are complementary to mRNA encoding galectin-3 are used to screen for polynucleotides in liver biopsy tissue, liver cell extracts, or other biological samples. RNA-detection-based diagnostic assays, such as Northern hybridization, Northern dot blots, RNA in situ hybridization, and ELISA assays, are particularly useful to distinguish between the absence, presence, and excess expression of galectins (e.g., galectin-3) and to monitor regulation of galectin levels during therapeutic intervention.

Included in the scope of the invention are the use of oligonucleotide sequences, antisense RNA and DNA molecules, and PNAs which complement galectin sequences, particularly galectin-3, for the determination of galectin expression in liver cells and tissue by RNA-based detection techniques. These forms of polynucleotide sequences encoding galectin-3 may also be used for the diagnosis of conditions or diseases with which the expression of galectin-3 is associated. For example, polynucleotide sequences complementary to mRNA encoding galectin-3 may be used in hybridization or PCR assays of fluids or tissues from biopsies to detect galectin-3 expression and thereby identify the subject as having a predisposition or established case of cirrhosis of the liver and/or HCC. The form of such qualitative and/or quantitative methods may include northern analysis, dot blot or other membrane-based technologies; PCR technologies; dip stick, pin, chip and ELISA technologies. All of these techniques are well known in the art and are the basis of many commercially available diagnostic kits.

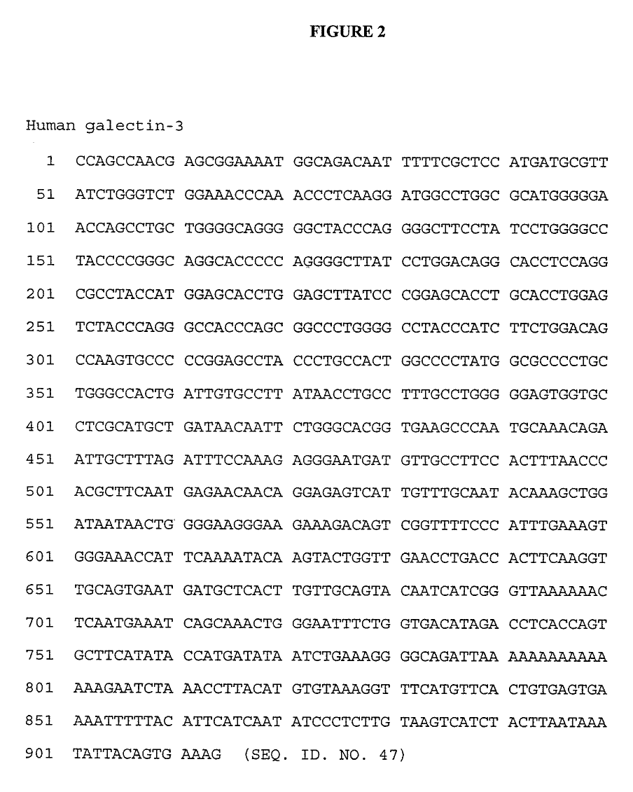

In one aspect, RNA probes complementary to galectin-3 mRNA are used in assays that detect activation or induction associated with disease (including cirrhosis and HCC). Accordingly, the nucleotide or polypeptide sequences of galectin-3 (referenced in Robertson et al., Biochemistry 29: 8093-8100 (1990) or Genbank Accession J02921, M57710, 4504982, or NID g4504982, g179530, NM_{—}002306.1 and NP_{—}002297), the disclosure of which are incorporated herein by reference in their entireties, is used to design suitable RNA probes. (See FIG. 2). The RNA probes are labeled by methods known in the art and are added to a DNAse treated fluid or tissue sample from a patient under conditions suitable for the formation of hybridization complexes. Hybridization on complexes are isolated or the sample is treated with an agent which removes unhybridized nucleic acids. After an incubation period, the sample is washed with a compatible fluid which optionally contains a dye (or other label requiring a developer) if the nucleotide has been labeled with an enzyme. After the compatible fluid is rinsed off, the dye is quantitated and compared with a standard. If the amount of dye in the biopsied or extracted sample is significantly elevated over that of a comparable control sample, the nucleotide sequence has hybridized with RNA in the sample, and the presence of elevated levels of RNA encoding galectin-3 in the sample indicates the presence of liver disease.

Such assays may also be used to evaluate the efficacy of a particular therapeutic treatment regime in animal studies, in clinical trials, or in monitoring the treatment of an individual patient. In order to provide a basis for the diagnosis of disease, a normal or standard profile for human galectin-3 expression in liver cells, extracts or tissue must be established. This is accomplished by combining body fluids or cell extracts taken from normal subjects with RNA probes encoding galectin-3, or a portion thereof, under conditions suitable for hybridization. Standard hybridization may be quantified by comparing the values obtained for normal subjects with a dilution series of galectin-3 RNA run in the same experiment where a known amount of substantially purified galectin-3 RNA is used. Standard values obtained from normal samples are then compared with values obtained from samples from subjects thought to be afflicted with liver disease. Deviation between standard and subject values establishes the presence of disease.

Additionally, PCR methods which may be used to quantitate the expression of a particular molecule include radiolabeling (Melby P. C. et al. J Immunol Methods 159:235-44 (1993)) or biotinylating nucleotides (Duplaa C. et al. Anal Biochem 212:229-236 (1993)), coamplification of a control nucleic acid, and standard curves onto which the experimental results are interpolated. Quantitation of multiple samples may be speeded up by running the assay in an ELISA format where the oligomer of interest is presented in various dilutions and a spectrophotometric or colorimetric response gives rapid quantitation. A definitive diagnosis of this type may allow health professionals to begin aggressive treatment and prevent further worsening of the condition. Similarly, further assays can be used to monitor the progress of a patient during treatment.

Once disease is established, a therapeutic agent is administered and a treatment profile is generated. Such assays may be repeated on a regular basis to evaluate whether the values in the profile progress toward or return to the normal or standard pattern. Successive treatment profiles may be used to show the efficacy of treatment over a period of several days or several months.

As mentioned above, PCR technology may be used to identify and quantitate expression of galectins, particularly galectin.3. For amplification of mRNAs, it is within the scope of the present invention to reverse transcribe mRNA into cDNA followed by PCR (RT-PCR); or, to use a single enzyme for both steps as described in U.S. Pat. No. 5,322,770, the disclosure of which is incorporated herein by reference in its entirety, or, to use Reverse Transcriptase Asymmetric Gap Ligase Chain Reaction (RT-AGLCR), as described by Marshall R. L. et al. (PCR Methods and Applications 4:80-84, 1994), the disclosure of which is incorporated herein by reference in its entirety.

A variety of PCR techniques are familiar to those skilled in the art. For a review of PCR technology, see Molecular Cloning to Genetic Engineering White, B. A. Ed. in Methods in Molecular Biology 67: Humana Press, Totowa (1997), the disclosure of which is incorporated herein by reference in its entirety and the publication entitled “PCR Methods and Applications” (1991, Cold Spring Harbor Laboratory Press), the disclosure of which is incorporated herein by reference in its entirety. In each of these PCR procedures, PCR primers on either side of the galectin-3 sequence to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188, the disclosure of which is incorporated herein by reference in its entirety.

The primers are selected to be substantially complementary to a portion of the sequence of galecting-3 mRNA and a portion of the sequence complementary to the sequence of galectin-3 mRNA, thereby allowing the sequences between the primers to be amplified. The length of the primers for use with aspects of the present invention can range from 8 to 100 nucleotides, preferably from 8 to 50, 8 to 30 or more preferably 8 to 25 nucleotides. Shorter primers tend to lack specificity for a target nucleic acid sequence and generally require cooler temperatures to form sufficiently stable hybrid complexes with the template. Longer primers are expensive to produce and can sometimes self-hybridize to form hairpin structures. The formation of stable hybrids depends on the melting temperature (Tm) of the DNA. The Tm depends on the length of the primer, the ionic strength of the solution and the G+C content. The higher the G+C content of the primer, the higher is the melting temperature because G:C pairs are held by three H bonds whereas A:T pairs have only two. The G+C content of the amplification primers of the present invention preferably ranges between 10 and 75%, more preferably between 35 and 60%, and most preferably between 40 and 55%. The appropriate length for primers under a particular set of assay conditions may be empirically determined by one of skill in the art.

The spacing of the primers determines the length of the segment to be amplified. In the context of the present invention amplified segments carrying nucleic acid sequence encoding a galectin can range in size from at least about 25 bp to 35 kbp. Amplification fragments from 25-3000 bp are typical, fragments from 50-1000 bp are preferred and fragments from 100-600 bp are highly preferred. It will be appreciated that amplification primers for a specific galectin may be any sequence which allows the specific amplification of any DNA fragment carrying nucleic acid sequence unique to the particular galectin. Amplification primers may be labeled or immobilized on a solid support as described above.

In the following discussion, we provide methods and compositions which are used to detect patterns of galectin expression in a biological sample so that the predisposition to disease can be determined.

Detection of patterns of galectin expression to identify predisposition to disease

Another aspect of the present invention is a method of correlating the ratio between the expression levels of a plurality of galectins with a disease state comprising obtaining biological samples from individuals suffering from the disease and normal individuals, determining the expression levels of two or more galectins in the samples, and determining whether there is a statistically significant association between the ratio of galectin expression and the disease state. Statistically significant associations can be determined using statistical methods familiar to those skilled in the art, including chi-squared analyses. In some embodiments, the disease state is cancer. Galectin expression may be measured using any of the methods described above for detecting galecting-3 protein or mRNA. Preferably, the galectins whose expression levels are measured to determine the ratios are selected from the group consisting of galectins 1-10. Alternatively, the galectins may be galectins which are currently unknown but which are identified based on their possession of one or more of the homology regions presented in Table III. Table III shows examples of conserved galectin residues from members of different animal species; the conserved residues are boxed. Similar patterns of amino acid conservation in context of each other can be used to differentiate galectin homologs in one animal species. Preferably, galectin expression is measured using the protein-based or nucleic acid based detection methods described above for galectin-3.

The overexpression, underexpression, or, sometimes, any expression of members of the galectin family is associated with several disease states and most acutely cancer. Galectin-1 and galectin-3 expression, for example, have been associated with tumor metastasis. (Raz et al., Cancer Res. 46:3667(1986) and Raz et al., Int. J. Cancer 46:871 (1990). Galectin 5 is thought to mediate cell adhesion and galectin-5H is believed to be involved with the onset of inflammatory disease and several forms of neoplastic disease. (Hillman et al., U.S. Pat. No. 5,837,493). The expression of galectin-7 has been observed to be downregulated in a malignant keratinocyte cell line and is thought to be required for the maintenance of normal keratinocytes. (Hillman et al., U.S. Pat. No. 5,837,493). The appearance of galectin 8 and 9 have also been associated with the onset of cancer. In the table below, several tumors that have been associated with the expression of various galectins are listed.

TABLE II
Association of Galectins with Neoplasms

| | | Normal | |
|---|---|---|---|
| Tumor Type | Galectin | Tissue Expression | Properties |
| Bladder | 1 | Expressed | Upregulated in |
| carcinoma | | | high-grade tumors |
| Anaplastic large | 3 | Absent in | Tumor marker |
| cell lymphoma | | T lymphocytes | |
| Colorectal | 3 | Expressed | Downregulated in |
| carcinoma | | | adenocarcinoma |
| Colorectal | 3 | Nuclear and | Conversion from |
| carcinoma | | cytoplasmic | nuclear to |
| | | | cytoplasmic with |
| | | | tumor progression |
| Ovarian carcinoma | 3 | Expressed | Downregulated in |
| | | | carcinoma |
| Breast carcinoma | 3 | Expressed | Downregulated in |
| | | | carcinoma |
| Thyroid carcinoma | 3 | Expressed | Tumor marker |
| Colorectal | 4 | Expressed | Down regulated |
| carcinoma | | | |
| Various | 7 | Expressed in | Downregulation |
| | | stratified epithelia | antigen |
| | | | Metaplasiogenic |
| | | | marker |
| Prostate | 8 | Absent | Tumor marker |
| Carcinoma | | | |
| Hodgkin's | 9 | Absent | Tumor marker |
| lymphoma | | | |

Because the family of galectins share several conserved domains, the elucidation of many more galectin family members will be forthcoming. (See Table 3). Many more tumor markers and/or markers for other forms of disease will be found.

TABLE III

| | | |
|---|---|---|
| | | 1 50 |
| | cegal | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| | eelga1 | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| (Seq. ID No.1) | chgal3 | MQAMKARCWQ PHWMLPLLFL SSPLHPQLSD ALPAHNPGAP PPQGWNRPPG |
| | ragal4 | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| | mugal4 | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| | hugal1 | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| | hugal4 | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| | | 51 100 |
| | cegal | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| | eelgal | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| (Seq. ID No.2) | chgal3 | PGAFPAYPGY PGAYPGAPGP YFGAPGPHHG PPGPYPGGPP GPYPGGPPGP |
| | ragal4 | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| | mugal4 | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| | hugal1 | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| | hugal4 | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| | | 101 150 |
| (Seq. ID No.3) | cegal | ˜˜˜˜˜˜˜˜˜˜ ˜˜˜˜MSAEEP KSYP..VPYR SVLQEKF... ....EPGQTL |
| (Seq. ID No.4) | eelgal | ˜˜˜˜˜˜˜˜˜˜ ˜˜˜˜˜˜˜˜˜˜ ˜˜˜˜˜˜˜˜˜˜ ˜˜˜SGGLQVK NFDFTVGKFL |
| (Seq. ID No.5) | chgal3 | YPGGPPGPYP GGPTAPYSEA PAAPLKVPYD LPLPAGLMPR LL.......I |
| (Seq. ID No.6) | ragal4 | ˜˜˜˜˜˜˜˜˜M AYVPAPGYQP TYNP.TLPYK RPIPGGL... ....SVGMSI |
| (Seq. ID No.7) | mugal4 | ˜˜˜˜˜˜˜˜˜˜ ˜˜˜˜˜˜˜˜˜˜ ˜˜˜˜˜˜˜˜˜˜ ˜PIPGGL... ....SVGMSV |
| (Seq. ID No.8) | hugal1 | ˜˜˜˜˜˜˜˜˜˜ ˜˜˜˜˜˜˜˜˜˜ ˜˜˜˜˜˜˜˜˜˜ ˜˜MACGLVAS NLNLKPGECL |
| (Seq. ID No.9) | hugal4 | ˜˜˜˜˜˜˜˜˜M AYVPAPGYQP TYNP.TLPYY QPIPGGL... ....NVGMSV |
| | | 151 200 |
| (Seq. ID No.10) | cegal | IVKGSTID.. ESQRFTINLH SKTADFSGND VPLHVSVRFD E.G...K..I |
| (Seq. ID No.11) | eelgal | TVGGFINNSP ..QRFSVN.. .VGESM..NS LSLHLDHRFN .YGAD.QNTI |
| (Seq. ID No.12) | chgal3 | TITGTVNSNP N..RFSLDFK R.GQD..... IAFNFNPRFK E...DHKRVI |
| (Seq. ID No.13) | ragal4 | YIQGIAKD.. NMRRFHVNF. AVGQD.EGAD IAFHFNPRFD ..GWD.K..V |
| (Seq. ID No.14) | mugal4 | YIQGMAKE.. NMRRFHVNF. AVGQD.DGAD VAFHFNPRFD ..GWD.K..V |
| (Seq. ID No.15) | hugal1 | RVRGEV..AP DAKSFVLN.. .LGKDS..NN LCLHFNPRFN AHG.D.ANTI |
| (Seq. ID No.16) | hugal4 | YIQGVASE.. HMKRFFVNF. VVGQD.PGSD VAFHFNPRFD ..GWD.K..V |
| | | 201 250 |
| (Seq. ID No.17) | cegal | VLNSF...SN GEWGKEERK. .SNPIKKGDS FDIRIRANDD RFQI.IVDHK |
| (Seq. ID No.18) | eelgal | VNNSTLKGDN G.WETEQRST .NFTLSAGQY FEITLSYDIN KFYIDILDGP |
| (Seq. ID No.19) | chgal3 | VCNSMF..QN N.WGKEERTA PRFPFEPGTP FKLQVLCEGD HFKVAV.NDA |
| (Seq. ID No.20) | ragal4 | VFNTM...QS GQWGKEEKKK .SMPFQKGHH FELVFMVMSE HYKV.VVNGT |
| (Seq. ID No.21) | mugal4 | VFKTM...QS GQWGKEEKKK .SMPFQKGKH FELVFMVMPE HYKV.VVNGN |
| (Seq. ID No.22) | hugal1 | VCNS..K.DG GAWGTEQREA .VFPFQPGSV AEVCITFDQA NLTVKLPDGY |
| (Seq. ID No.23) | hugal4 | VFNTL...QG GKWGSEERKR .SMPFKKGAA FELVFIVMAE HYKV.VVNGN |
| | | 251 300 |
| (Seq. ID No.24) | cegal | EFKDYEHR.L PLSSISHLSI DGDLYLNNV. .HW.GGK... ..Y....... |
| (Seq. ID No.25) | eelgal | NL.EFPNR.Y SKEFLPFLSL AGDARLTLV. .K.E˜˜˜˜˜˜ ˜˜˜˜˜˜˜˜˜˜ |
| (Seq. ID No.26) | chgal3 | HLLQFNFREK KLNGITKLCI AGDITLTSVL TSMI˜˜˜˜˜ ˜˜˜˜˜˜˜˜˜˜ |
| (Seq. ID No.27) | ragal4 | PFYEYGHR.L PLQMVTHLQV DGDLELQSI. .NFLGGQPAA SQYFGTMTIP |
| (Seq. ID No.28) | mugal4 | SFYEYGHR.L PVQMVTHLQV DGDLELQSI. .NFLGGQPAA APYAGAMTIP |
| (Seq. ID No.29) | hugal1 | EF.KFFNR.L NLEAINYMAA DGDFKIKCV. .AFD˜˜˜˜˜ ˜˜˜˜˜˜˜˜˜˜ |
| (Seq. ID No.30) | hugal4 | PFYEYGHR.L FLQMVTHLQV DGDLQLQSI. .NFIGGQPLR PQ..GPPMMP |
| | | 301 350 |
| (Seq. ID No.31) | cegal | .YP....... .......... .........V PYESGLANGL PVGKSLLVFG |
| | eelgal | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| | chgal3 | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| (Seq. ID No.32) | ragal4 | AYP..SAGYN PPQMNSLPVM AGPPIFNPPV PYVGTLQGGL TARRTIIIKG |
| (Seq. ID No.33) | mugal4 | AYPAGSPGYN PPQMNTLPVM TGPPVFNPRV PYVGALQGGL TLPRTIIIKG |
| | hugall | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| (Seq. ID No.34) | hugal4 | PYP..GPGHC HQQLNSLPTM EGPPTFNP.V PYFGRLQGGL TARRTIIIKG |
| | | 351 400 |
| (Seq. ID No.35) | cegal | TVEKKAKRFH VNL.LRKNGD ISFHFNPRFD EKHVIRNSLA ANEWGNEERE |
| | eelgal | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| | chgal3 | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| (Seq. ID No.36) | ragal4 | YVLPTAKNLI INFKVGSTGD IAFHMNPRIG D.CVVRNSYM NGSWGSEERK |
| (Seq. ID No.37) | mugal4 | YVLPTARNFV INFKVGSSGD IALHLNPRIG D.SVVFNSFM NGSWGAEERK |
| | hugal1 | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| (Seq. ID No.38) | hugal4 | YVPPTGKSFA INFKVGSSGD IALHINPH.G NGTVVRNSLL NGSWGSEEKK |
| | | 401 450 |
| (Seq. ID No.39) | cegal | .GKNPFEKGV G..FDLVIQN EEYAFQVFVN GERYISFAHR ADPHD.IAGL |
| | eelgal | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| | chgal3 | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| (Seq. ID No.40) | ragal4 | IPYNPF..GA GQFFDLSIRC GTDRFKVFAN GQHLFDFSHR FQAFQRVDML |
| (Seq. ID No.41) | mugal4 | VAYNPF..GP GQFFDLSIRC GMDRFKVFAN GQHLFDFSHR FQAFQMVDTL |
| | hugal1 | ˜˜˜˜˜˜˜˜˜˜ ˜˜˜˜˜˜˜˜˜˜ ˜˜˜˜˜˜˜˜˜˜ ˜˜˜˜˜˜˜˜˜˜ ˜˜˜˜˜˜˜˜˜˜ |
| (Seq. ID No.42) | hugal4 | TTHNFF..GP GQFFDLSIRC GLDRFKVYAN GQHLFDFAHP SRAFQRVDTL |
| | | 451 465 |
| (Seq. ID No.43) | cegal | QISGDIELSG IQIQ |
| | eelgal | ˜˜˜˜˜˜˜˜˜˜ ˜˜˜˜˜ |
| | chgal3 | ˜˜˜˜˜˜˜˜˜˜ ˜˜˜˜˜ |
| (Seq. ID No.44) | ragal4 | EIKGDITLSY VQI˜ |
| (Seq. ID No.45) | mugal4 | EINGDITL˜˜ ˜˜˜˜˜ |
| | hugal1 | ˜˜˜˜˜˜˜˜˜˜ ˜˜˜˜˜ |
| (Seq. ID No.46) | hugal4 | EIQGDVTLSY VQI˜˜ |

| | |
|---|---|
| (Seq. ID No.47) | DNA Human galectin-3 |
| | CCAGCCAACGAGCGGAAAATGGCAGACAATTTTTCGCTCCATGATGCGTTATCTGGGTCT |
| | GGAAACCCAAACCCTCAAGGATGGCCTGGCGCATGGGGGAACCAGCCTGCTGGGGCAGGG |
| | GGCTACCCAGGGGCTTCCTATCCTGGGGCCTACCCCGGGCAGGCACCCCCAGGGGCTTAT |
| | CCTGGACAGGCACCTCCAGGCGCCTACCATGGAGCACCTGGAGCTTATCCCGGAGCACCT |
| | GCACCTGGAGTCTACCCAGGGCCACCCAGCGGCCCTGGGGCCTACCCATCTTCTGGACAG |
| | CCAAGTGCCCCCGGAGCCTACCCTGCCACTGGCCCCTATGGCGCCCCTGCTGGGCCACTG |
| | ATTGTGCCTTATAACCTGCCTTTGCCTGGGGGAGTGGTGCCTCGCATGCTGATAACAATT |
| | CTGGGCACGGTGAAGCCCAATGCAAACAGAATTGCTTTAGATTTCCAAAGAGGGAATGAT |
| | GTTGCCTTCCACTTTAACCCACGCTTCAATGAGAACAACAGGAGAGTCATTGTTTGCAAT |
| | ACAAAGCTGGATAATAACTGGGGAAGGGAAGAAAGACAGTCGGTTTTCCCATTTGAAAGT |
| | GGGAAACCATTCAAAATACAAGTACTGGTTGAACCTGACCACTTCAAGGTTGCAGTGAAT |
| | GATGCTCACTTGTTGCAGTACAATCATCGGGTTAAAAAACTCAATGAAATCAGCAAACTG |
| | GGAATTTCTGGTGACATAGACCTCACCAGTGCTTCATATACCATGATATAATCTGAAAGG |
| | GGCAGATTAAAAAAAAAAAAAAAGAATCTAAACCTTACATGTGTAAAGGTTTCATGTTCA |
| | CTGTGAGTGAAAATTTTTACATTCATCAATATCCCTCTTGTAAGTCATCTACTTAATAAA |
| | TATTACAGTGAAAG |

cegal — nematode
eelgal — eel
chgal3 — chicken
ragal4 — rat
mugal4 — mouse
hugall — human
hugal4 — human

In another aspect of the present invention, we contemplate the identification of patterns of galectin expression so as to identify a disease state. When the expression of all members of the galectin family are analyzed, a predisposition for various diseases can be ascertained. In particular, the complete expression pattern of the galectin family in all normal and tumor tissues will indicate both pre-neoplastic and post-neoplastic events.

By one approach, diagnostic tools which simultaneously screen for all galectin family members are developed. Current technology, such as gene array scanning systems permits the execution of our strategy due to its inherent large-scale screening abilities, accuracy, generation of quantitative information and efficiency in both preparation of diagnostic arrays and data scanning and accumulation. Accordingly, the approaches to monitoring the expression of galectin-3, detailed above, (e.g., the protein and RNA detection methods) can be modified so that the expression of several galectins from numerous tissues or biological sample sources can be simultaneously analyzed. The use of traditional techniques, such as antibody-mediated serum assays, immunohistochemistry, ELISA, genechip northerns, and PCR will permit us to screen for multiple galectins rapidly. Additionally, the quantity of each galectin is preferably determined. The amount of RNA or protein corresponding to the various galectins can be quantified by the approaches detailed above or by many other methods known to those of skill in the art. For example, the total protein or RNA in a sample is first determined by conventional techniques (e.g., by spectroscopy), and then by using a probe having a label with a known specific activity, the amount of galectin expression can be accurately determined and normalized to a “house keeping marker.”

Once the levels of the various galectins are determined, the information is recorded onto a computer readable media, such as a hard drive, floppy disk, DVD drive, zip drive, etc.. After recording and the generation of a database comprising the levels of expression of the various galectins studied, a comparing program is used which compares the levels of expression of the various galectins so as to create a ratio of expression. In a first comparison, a galectin to galectin ratio is generated. For example, desirable galectin to galectin ratios include, but are not limited to: galectin-1:galectin-3, galectin-1:galectin-5, galectin-1:galectin-7, galectin-1:galectin-8, galectin-1:galectin-9, galectin-3:galectin-5, galectin-3:galectin-7, galectin-3:galectin-8, galectin-3:galectin-9, galectin-5:galectin-7, galectin-5:galectin-8, galectin-5:galectin 9, galectin-7:galectin-8, galectin-7:galectin-9, and galectin-8:galectin-9. As other newly found galectins are identified, we contemplate screening for their expression patterns and incorporating the levels of expression into our ratio analysis approach to disease prognosis. In a second comparison, the galectin to galectin ratios from normal subjects are compared to galectin to galectin ratios of subjects having various diseases. Preferably, the diseased subjects studied initially have been identified as having a form of cancer. Desirably, several databases are generated comprising the galectin to galectin ratios from normal individuals and the galectin to galectin ratios from diseased subjects so that a statistical analysis can be accurately performed. In this manner patterns of galectin expression are analyzed and the predisposition to galectin related disease is determined.

Individuals who wish to be screened for a predisposition for a galectin related disease can have a biological sample taken, and the RNA or protein present in the sample anlayzed for the galectin expression pattern, and the galectin to galectin ratios are compared to galectin to galectin ratios exhibited by both normal and diseased subjects. Depending on the comparison of the normal and diseased ratios, a helath care practitioner can choose an appropriate course of treatment and/or prevention.

In the following discussion, we provide methods and compositions which are used to treat and/or prevent diseases associated with increased levels of galectin expression.

Therapeutic and prophylactic agents

In addition to the diagnosis of liver disease and galectin-3 related diseases, galectin-3 -specific antibodies are useful for the treatment of conditions and diseases associated with expression of galectin-3. Such antibodies include, but are not limited to, polyclonal, monoclonal, chimeric, single chain, Fab fragments and fragments produced by a Fab expression library. Neutralizing antibodies, i.e., those which inhibit dimer formation, are especially preferred for diagnostics and therapeutics.

Galectin-3 protein to be used for antibody production need not retain biological activity; however, the protein fragment, or oligopeptide is desirably antigenic. Peptides used to induce specific antibodies may have an amino acid sequence consisting of at least five amino acids, preferably at least 10 amino acids. Preferably, they should mimic a portion of the amino acid sequence of the natural protein and may contain the entire amino acid sequence of a small, naturally occurring molecule. Short stretches of galectin-3 amino acids may be fused with those of another protein such as keyhole limpet hemocyanin and antibody produced against the chimeric molecule.

For the production of antibodies, various hosts including goats, rabbits, rats, mice, etc may be immunized by injection with galectin-3 or any portion, fragment or oligopeptide which retains immunogenic properties. Depending on the host species, various adjuvants may be used to increase immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (Bacillus Calmette-Guerin) and Corynebacterium parvum are potentially useful adjuvants.

Monoclonal antibodies to galectin-3 may be prepared using any technique which provides for the production of antibody molecules by continuous cell lines in culture. These include but are not limited to the hybridoma technique originally described by Koehler and Milstein (Nature 256:495-497 (1975), the human B-cell hybridoma technique (Kosbor et al. Immunol Today 4:72 (1983); Cote et al Proc Natl Acad Sci 80:2026-2030 (1983), and the EBV-hybridoma technique Cole et al. Monoclonal Antibodies and Cancer Therapy, Alan R. Liss Inc, New York N.Y., pp 77-96 (1985).

In addition, techniques developed for the production of “chimeric antibodies”, the splicing of mouse antibody genes to human antibody genes to obtain a molecule with appropriate antigen specificity and biological activity can be used. (Morrison et al. Proc Natl Acad Sci 81:6851-6855 (1984); Neuberger et al. Nature 312:604-608(1984); Takeda et al. Nature 314:452-454(1985). Alternatively, techniques described for the production of single chain antibodies (U.S. Pat. No. 4,946,778) can be adapted to produce galectin-3 -specific single chain antibodies.

Antibodies may also be produced by inducing in vivo production in the lymphocyte population or by screening recombinant immunoglobulin libraries or panels of highly specific binding reagents as disclosed in Orlandi et al., Proc Natl Acad Sci 86: 3833-3837 (1989), and Winter G. and Milstein C; Nature 349:293-299 (1991).

Antibody fragments which contain specific binding sites for galectin-3 may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity Huse W. D. et al. Science 256:1275-1281 (1989).

By “antibody therapy” is meant the administration of an antibody, an antibody conjugate or an antibody heteroconjugate to a subject in need for the purpose of treating or preventing a disease. Many forms of antibody therapy are known in the art including, but not limited to, the administration of monoclonal or humanized monoclonal antibodies, the administration of toxin or radionuclide conjugated antibodies, and the administration of monoclonal antibody heteroconjugates having one domain that binds to a disease-related antigen (e.g., a galectin such as galectin-3) and another domain that binds to the Fc region of IgG. Further, the term antibody therapy is meant to include the administration of (Fab′)₂ or Fab fragments with or without conjugated toxins or radionuclides. Several examples of antibody therapy are found in Recent Results in Cancer Research vol 141:Systemic Radiotherapy with Monoclonal Antibodies. edited by M. L. Sautter-Bihl and M. Wannenmacher, Springer- Verlag publishers, 1996.

Radiolabelled mAbs specific for a tumor antigen (e.g., a galectin such as galectin-3) are prepared by labeling the antibody with an isotope or combinations of isotopes, such as ¹³¹I, ⁹⁰Y, ⁶⁷Cu, ¹⁸⁶Re, ¹⁸⁸Re, ²¹²Bi or ²¹¹At. Preferable radiolabeled mAbs are able to deliver more than 6000 rads to the tumor and have sufficient affinity so that the patient's bone marrow is not exposed to more than 300 rads. We contemplate the use of ¹³¹I labeled anti-galectin-3, as well as other radiolabeled mAbs for treatment of HCC and cirrhosis of the liver. In this aspect and in several other embodiments using radionuclide or toxin conjugated antibodies, a delivery system which preferentially allows delivery of the antibody therapy agent to diseased cells is desired. By one approach, a liposome having the antibody therapy agent and a membrane bound protein which interacts specifically with liver cells is used. Additionally, the localized delivery of the antibody therapy agent (e.g., injecting the agent directly into liver tissue) is contemplated.

Further, the development of peptidomimetics and other compounds which interfere with galectin-3 function so as to generate pharmaceuticals which can be used to treat galectin-3 related liver diseases are embodiments of the invention. Galectin-3, its catalytic or immunogenic fragments or oligopeptides thereof, can be used for screening therapeutic compounds in any of a variety of drug screening techniques. The fragment employed in such a test may be free in solution, affixed to a solid support, borne on a cell surface, or located intracellularly. The formation of binding complexes, between human galectin-3 and the agent being tested, may be measured. Another technique for drug screening which may be used for high throughput screening of compounds having suitable binding affinity to galectin-3 is described in detail in “Determination of Amino Acid Sequence Antigenicity” by Geysen H. N., WO Application 84/03564, published on Sep. 13, 1984, and incorporated herein by reference. In summary, large numbers of different small peptide test compounds are synthesized on a solid substrate, such as plastic pins or some other surface. The peptide test compounds are reacted with fragments galectin-3 and washed. Bound galectin-3 is then detected by methods well known in the art. Substantially purified galectin-3 can also be coated directly onto plates for use in the aforementioned drug screening techniques. Alternatively, non-neutralizing antibodies can be used to capture the peptide and immobilize it on a solid support or many of the approaches for attaching a biomolecule to a support, described above, can be used. Once test compounds are found, they are preferably tested in a test subject having liver disease such as HCC and cirrhosis of the liver and the effect on galectin-3 activity is analyzed by one or more of the approaches detailed above. Successful agents are then incorporated into pharmaceuticals and are used to treat and/or prevent liver diseases such as HCC and cirrhosis of the liver.

Additionally, we contemplate the use of antisense and ribozyme technology to inhibit the expression of galectin-3 and/or other galectins. The antisense nucleic acid molecules to be used in gene therapy may be either DNA or RNA sequences. They may comprise a sequence complementary to the sequence of mRNA encoding galectin-3. The antisense nucleic acids should have a length and melting temperature sufficient to permit formation of an intracellular duplex having sufficient stability to inhibit the expression of the mRNA in the duplex. Strategies for designing antisense nucleic acids suitable for use in gene therapy are disclosed in Green et al., Ann. Rev. Biochem., 55:569-597 (1986) and Izant and Weintraub, Cell, 36:1007-1015 (1984).

In some strategies, antisense molecules are obtained from a nucleotide sequence encoding galectin-3 by reversing the orientation of the coding region with respect to a promoter so as to transcribe the opposite strand from that which is normally transcribed in the cell. Antisense molecules and ribozymes may be prepared by any method known in the art for the synthesis of RNA molecules. These include techniques for chemically synthesizing oligonucleotides such as solid phase phosphoramidite chemical synthesis. Alternatively, RNA molecules may be generated by in vitro and in vivo transcription of DNA sequences encoding galectin-3 or another galectin. Such DNA sequences may be incorporated into a wide variety of vectors with suitable RNA polymerase promoters such as T7 or SP6. Alternatively, antisense cDNA constructs that synthesize antisense RNA constitutively or inducibly can be introduced into cell lines, cells or tissues.

Alternatively, oligonucleotides which are complementary to the mRNA encoding galectin-3 may be synthesized in vitro. Thus, antisense nucleic acids are capable of hybridizing to the galectin-3 mRNA to create a duplex. In some embodiments, the antisense sequences may contain modified sugar phosphate backbones to increase stability and make them less sensitive to RNase activity. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine and wybutosine as well as acetyl-, methyl-, thio- and similarly modified forms of adenine, cytidine, guanine, thymine, and uridine which are not as easily recognized by endogenous endonucleases. Further examples are described by Rossi et al., Pharmacol. Ther., 50(2):245-254, (1991).

Various types of antisense oligonucleotides complementary to the galectin-3 mRNA may be used. In one preferred embodiment, stable and semi-stable antisense oligonucleotides described in International Application No. PCT WO94/23026, hereby incorporated by reference, are used. In these moleucles, the 3□ end or both the 3□ and 5□ ends are engaged in intramolecular hydrogen bonding between complementary base pairs. These molecules are better able to withstand exonuclease attacks and exhibit increased stability compared to conventional antisense oligonucleotides. In another preferred embodiment, the antisense oligodeoxynucleotides described in International Application No. WO 95/04141 are used.

In yet another preferred embodiment, the covalently cross-linked antisense oligonucleotides described in International Application No. WO 96/31523 are used. These double- or single-stranded oligonucleotides comprise one or more, respectively, inter- or intra-oligonucleotide covalent cross-linkages, wherein the linkage consists of an amide bond between a primary amine group of one strand and a carboxyl group of the other strand or of the same strand, respectively, the primary amine group being directly substituted in the 2′ position of the strand nucleotide monosaccharide ring, and the carboxyl group being carried by an aliphatic spacer group substituted on a nucleotide or nucleotide analog of the other strand or the same strand, respectively.

The antisense oligodeoxynucleotides and oligonucleotides disclosed in International Application No. WO 92/18522 may also be used. These molecules are stable to degradation and contain at least one transcription control recognition sequence which binds to control proteins and are effective as decoys therefor. These molecules may contain “hairpin” structures, “dumbbell” structures, “modified dumbbell” structures, “cross-linked” decoy structures and “loop” structures. In another preferred embodiment, the cyclic double-stranded oligonucleotides described in European Patent Application No. 0 572 287 A2 are used. These ligated oligonucleotide “dumbbells” contain the binding site for a transcription factor and inhibit expression of the gene under control of the transcription factor by sequestering the factor. Use of the closed antisense oligonucleotides disclosed in International Application No. WO 92/19732, hereby incorporated by reference, is also contemplated. Because these molecules have no free ends, they are more resistant to degradation by exonucleases than are conventional oligonucleotides. These oligonucleotides may be multifunctional, interacting with several regions which are not adjacent to the target mRNA.

The appropriate level of antisense nucleic acids required to inhibit gene expression may be determined using in vitro expression analysis. The antisense molecule may be introduced into the cells expressing galectin-3 by diffusion, injection, infection or transfection using procedures known in the art. For example, the antisense nucleic acids can be introduced into the body as a bare or naked oligonucleotide, oligonucleotide encapsulated in lipid, oligonucleotide sequence encapsidated by viral protein, or as an oligonucleotide operably linked to a promoter contained in an expression vector. The expression vector may be any of a variety of expression vectors known in the art, including retroviral or viral vectors, vectors capable of extrachromosomal replication, or integrating vectors. The vectors may be DNA or RNA.

The antisense molecules are introduced onto cell samples at a number of different concentrations preferably between 1×10⁻¹⁰M to 1×10⁻⁴M. Once the minimum concentration that can adequately control gene expression is identified, the optimized dose is translated into a dosage suitable for use in vivo. For example, an inhibiting concentration in culture of 1×10⁻⁷ translates into a dose of approximately 0.6 mg/kg bodyweight. Levels of oligonucleotide approaching 100 mg/kg bodyweight or higher may be possible after testing the toxicity of the oligonucleotide in laboratory animals. It is additionally contemplated that cells from a vertebrate, such as a mammal or human, are removed, treated with the antisense oligonucleotide, and reintroduced into the vertebrate.

Ribozymes may also be used to reduce or eliminate galectin-3 expression. Ribozymes are enzymatic RNA molecules capable of catalyzing the specific cleavage of RNA. The mechanism of ribozyme action involves sequence-specific hybridization of the ribozyme molecule to complementary target RNA, followed by endonucleolytic cleavage. Within the scope of aspects of the invention, are engineered hammerhead motif ribozyme molecules that can specifically and efficiently catalyze endonucleolytic cleavage of a sequence encoding human galectin-3, for example. Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays. Delivery of antisense and ribozyme agents by transfection and by liposome are quite well known in the art.

The pharmacologically active compounds of this invention can be processed in accordance with conventional methods of galenic pharmacy to produce medicinal agents for administration to patients, e.g., mammals including humans. The antibodies, fragments thereof, peptidomimetics, antisense, and ribozymes may be incorporated into a pharmaceutical product with and/or without modification. Further, we envision the manufacture of pharmaceuticals or therapeutic agents which deliver anti-galectin-3 agents or a nucleic acid sequence encoding the anti-galectin-3 agent by several routes. For example, and not by way of limitation, the use of DNA, RNA, and viral vectors having sequence encoding an antibody to galectin-3 is contemplated. Nucleic acids encoding an antibody to galectin-3 can be administered alone or in combination with peptides or an antibody to galectin-3.

The compounds of this invention can be employed in admixture with conventional excipients, i.e., pharmaceutically acceptable organic or inorganic carrier substances suitable for parenteral, enteral (e.g., oral) or topical application which do not deleteriously react with the active compounds. Suitable pharmaceutically acceptable carriers include but are not limited to water, salt solutions, alcohols, gum arabic, vegetable oils, benzyl alcohols, polyetylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined where desired with other active agents, e.g., vitamins.

For parenteral application, particularly suitable are injectable, sterile solutions, preferably oily or aqueous solutions, as well as suspensions, emulsions, or implants, including suppositories. Ampoules are convenient unit dosages.

For enteral application, particularly suitable are tablets, dragees, liquids, drops, suppositories, or capsules. A syrup, elixir, or the like can be used wherein a sweetened vehicle is employed.

Sustained or directed release compositions can be formulated, e.g., liposomes or those wherein the active compound is protected with differentially degradable coatings, e.g., by microencapsulation, multiple coatings, etc. It is also possible to freeze-dry the new compounds and use the lyophilizates obtained, for example, for the preparation of products for injection.

For topical application, there are employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., a freon.

It will be appreciated that the actual preferred amounts of active compound in a specific case will vary according to the specific compound being utilized, the particular compositions formulated, the mode of application, and the particular situs and organism being treated. Pharmaceutical compositions suitable for use in the present invention include compositions wherein the active ingredients are contained in an effective amount to achieve the intended purpose. Dosages for a given host can be determined using conventional considerations, e.g., by customary comparison of the differential activities of the subject compounds and of a known agent, e.g., by means of an appropriate, conventional pharmacological protocol. The determination of an effective dose is well within the capability of those skilled in the art.

For any compound, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans.

A therapeutically effective dose refers to that amount of protein or its antibodies, antagonists, or inhibitors which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state, e.g., tumor size and location; age, weight and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 10 g, depending upon the route of administration. Guidance as to particular dosages and methods of delivery is provided in the literature. See U.S. Pat. Nos. 4,657,760; 5,206,344; or 5,225,212. Those skilled in the art will employ different formulations for nucleotides than for proteins or their inhibitors. Similarly, delivery of polynucleotides or polypeptides will be specific to particular cells, conditions, locations, etc..

Although the invention has been described with reference to embodiments and examples, it should be understood that various modifications can be made without departing from the spirit of the invention. Accordingly, the invention is limited only by the following claims. All references cited herein are hereby expressly incorporated by reference.

What is claimed is:

1. A method of identifying the presence of galectin-3 in cirrhotic or cancerous liver comprising:
obtaining a sample of cirrhotic or cancerous liver tissue;
providing an antibody to galectin-3;
contacting the sample with the antibody; and
analyzing the presence of galectin-3 in the sample.

2. The method of claim 1, wherein the cancerous liver tissue is hepatocellular carcinoma.

3. The method of claim 1, wherein the analysis of the presence of galectin-3 in the sample comprises a technique of the group consisting of fluorescence activated cell sorting (FACS), immunoprecipitation, Western blot, antibody staining, and a galectin-3 binding assay.

4. A method of identifying the amount galectin-3 expression in cirrhotic or cancerous liver comprising:
obtaining a sample of cirrhotic or cancerous liver tissue;
providing a probe which interacts with galectin-3 or an RNA encoding galectin-3;
contacting the sample with the probe; and
analyzing the presence of galectin-3 in the sample.

5. The method of claim 4, wherein the cancerous liver tissue is hepatocellular carcinoma.

6. The method of claim 4, wherein the probe is a member of the group consisting of a nucleic acid, a protein, a carbohydrate chemical agent, and a peptidomimetic.

7. The method of claim 4, wherein the analysis of the presence of galectin-3 in the sample comprises a technique of the group consisting of fluorescence activated cell sorting (FACS), immunoprecipitation, Western blot, antibody staining, a galectin-3 binding assay, and a hybridization assay.

8. The method of claim 4 further comprising quantifying the amount of galectin-3 protein or RNA in the sample.

9. The method of claim 4 or 8 further comprising recording the amount of galectin-3 expression or the amount of galectin-3 on a computer readable media.

10. A method of identifying a subject in need of treatment or prevention of liver disease comprising:
obtaining a biological sample from a subject having RNA or protein, wherein the biological sample is obtained from the subject's liver;
providing a probe which interacts with galectin-3 protein or RNA encoding galectin-3;
contacting the biological sample with the probe under conditions which allow the probe to interact with the RNA or protein in the biological sample;
determining the amount of probe which interacts with the RNA or protein in the biological sample so as to determine the amount of galectin-3 expression; and
identifying the subject as a subject in need of treatment or prevention of liver disease by the presence of the probe in hepatocytes.

11. The method of claim 10, wherein the liver disease is cirrhosis or hepatocellular carcinoma, or any neoplasm originating from the liver.

12. The method of claim 10, wherein the biological sample comprises an hepatocyte.

13. The method of claim 10, wherein the probe is a member of the group consisting of a nucleic acid, a protein, a carbohydrate chemical agent, and a peptidomimetic.

14. The method of claim 10, wherein the determination of the amount of probe which interacts with the RNA or protein comprises a technique of the group consisting of fluorescence activated cell sorting (FACS), immunoprecipitation, Western blot, antibody staining, a galectin-3 binding assay, and a hybridization assay.

15. The method of claim 10 further comprising quantifying the amount of galectin-3 protein or RNA in the sample.

16. The method of claim 10 or 15 further comprising recording the amount of galectin-3 expression on a computer readable media.

17. A method of making a pharmaceutical for the treatment or prevention of hepatocellular carcinoma comprising incorporating an agent which inhibits the production of galectin-3 in liver cells or tissue in a pharmaceutical formulation, wherein the agent is a member of the group consisting of an antibody to galectin-3, a peptide, a carbohydrate, a chemical agent, a peptidomimetic which interacts with galectin-3, an antisense oligonucleotide complementary to a transcript encoding galectin-3, and a ribozyme complementary to a transcript encoding galectin-3.

18. A method of treatment or production of hepatocellular carcinoma or cirrhosis of the liver comprising administering an agent which inhibits the production of galectin-3 in liver cells or tissue in a pharmaceutical formulation, wherein the agent is a member of the group consisting of an antibody to galectin-3, a carbohydrate, a chemical agent, a peptide a peptidomimetic which interacts with galectin-3, an antisense oligonucleotide complementary to a transcript encoding galectin-3, and a ribozyme complementary to a transcript encoding galectin-3.

19. The method of claims 17 or 18, wherein the agent is an antibody conjugated to a toxin or a radionuclide.

20. A method of screening for prognosis of cirrhosis or neoplasm originating from liver in a human comprising:
(a) isolating test proteins or RNA from a biological sample comprising hepatocytes, said biological sample being obtained from said subject; and
(b) determining the level of galectin-3 protein or an RNA encoding galectin-3 in said biological sample, wherein an elevated level of said galectin-3 protein or RNA encoding galectin-3 indicates a positive correlation with development of cancer or cirrhosis of the liver, and wherein the step of determining the level of galectin-3 protein or an RNA encoding galectin-3 is carried out by probing said galectin-3 protein or RNA encoding galectin-3 with an antibody which recognizes galectin-3 protein or a nucleic acid complementary to the RNA encoding galectin-3.

21. A method of screening for risk of cancer or cirrhosis of the liver in a human subject comprising:
(a) isolating test proteins or RNA from a biological sample comprising hepatocytes, said biological sample being obtained from said subject;
(b) determining the level of galectin-3 protein or an RNA encoding galectin-3 in said biological sample, wherein an elevated level of said galectin-3 protein or RNA encoding galectin-3 indicates a positive correlation with development of cancer or cirrhosis of the liver, and wherein the step of determining the level of galectin-3 protein or an RNA encoding galectin-3 is carried out by probing said galectin-3 protein or RNA encoding galectin-3 with an antibody which recognizes galectin-3 protein or a nucleic acid complementary to the RNA encoding galectin-3; and
(c) identifying the human subject as being at risk based on the level of galectin-3 protein or RNA encoding galectin-3 determined in step (b).

22. The method of claim 20 or 21 wherein said cancer is hepatocellular carcinoma or any neoplasm of liver origin.

23. A method for determining whether an individual is suffering from hepatocellular carcinoma or cirrhosis of the liver or is likely to suffer to suffer from hepatocellular carcinoma or any neoplasm of liver origin or cirrhosis of the liver in the future comprising determining whether the level of galectin-3 in the liver of said individual is above normal.

24. The method of claim 23, wherein the step of determining whether the level of galectin-3 expression in the liver of said individual is above normal comprises determining whether the level of galectin-3 protein in a biological sample obtained from the liver of said individual is above normal.

25. The method of claim 24, wherein the step of determining whether the level of galectin-3 protein in a biological sample obtained from the liver of said individual is above normal comprises:
contacting said biological sample with an antibody capable of specifically binding to galectin-3; and
determining whether the level of antibody binding to said sample is greater than the level of antibody binding observed in samples from individuals who are not suffering from hepatocellular carcinoma or cirrhosis of the liver.

26. The method of claim 25, wherein the biological sample is a liver biopsy.

27. The method of claim 26, wherein the determining step comprises determining whether the level of antibody binding to hepatocytes in the liver biopsy is greater than the level observed in biopsies from individuals not suffering from hepatocellular carcinoma or cirrhosis of the liver.

28. The method of claim 23, wherein the step of determining the whether the level of galectin-3 expression in the liver of said individual is above normal comprises determining the level of mRNA encoding galectin-3 present in a biological sample obtained from the liver of said individual.

29. The method of claim 28, wherein the level of galectin-3 mRNA is determined by a method from the group consisting of a nucleic acid amplification reaction, a nucleic acid hybridization assay, and an RNAse protection assay.


SEQUENCE LISTING (47 sequences)

SEQ ID NO: 1 - protein, 50 aa, chicken
MQAMKARCWQPHWMLPLLPLSSPLHPQLSDALPAHNPGAPPPQGWNRPPG

SEQ ID NO: 2 - protein, 50 aa, chicken
PGAFPAYPGYPGAYPGAPGPYPGAPGPHHGPPGPYPGGPPGPYPGGPPGP

SEQ ID NO: 3 - protein, 27 aa, nematode
MSAEEPKSYPVPYRSVLQEKFEPGQTL

SEQ ID NO: 4 - protein, 17 aa, eel
SGGLQVKNFDFTVGKFL

SEQ ID NO: 5 - protein, 43 aa, chicken
YPGGPPGPYPGGPTAPYSEAPAAPLKVPYDLPLPAGLMPRLLI

SEQ ID NO: 6 - protein, 33 aa, rat
MAYVPAPGYQPTYNPTLPYKRPIPGGLSVGMSI

SEQ ID NO: 7 - protein, 12 aa, mouse
PIPGGLSVGMSV

SEQ ID NO: 8 - protein, 18 aa, human
MACGLVASNLNLKPGECL

SEQ ID NO: 9 - protein, 33 aa, human
MAYVPAPGYQPTYNPTLPYYQPIPGGLNVGMSV

SEQ ID NO: 10 - protein, 42 aa, nematode
IVKGSTIDESQRFTINLHSKTADFSGNDVPLHVSVRFDEGKI

SEQ ID NO: 11 - protein, 41 aa, eel
TVGGFINNSPQRFSVNVGESMNSLSLHLDHRFNYGADQNTI

SEQ ID NO: 12 - protein, 39 aa, chicken
TITGTVNSNPNRFSLDFKRGQDIAFHFNPRFKEDHKRVI

SEQ ID NO: 13 - protein, 41 aa, rat
YIQGIAKDNMRRFHVNFAVGQDEGADIAFHFNPRFDGWDKV

SEQ ID NO: 14 - protein, 41 aa, mouse
YIQGMAKENMRRFHVNFAVGQDDGADVAFHFNPRFDGWDKV

SEQ ID NO: 15 - protein, 41 aa, human
RVRGEVAPDAKSFVLNLGKDSNNLCLHFNPRFNAHGDANTI

SEQ ID NO: 16 - protein, 41 aa, human
YIQGVASEHMKRFFVNFVVGQDPGSDVAFHFNPRFDGWDKV

SEQ ID NO: 17 - protein, 44 aa, nematode
VLNSFSNGEWGKEERKSNPIKKGDSFDIRIRAHDDRFQIIVDHK

SEQ ID NO: 18 - protein, 48 aa, eel
VMNSTLKGDNGWETEQRSTNFTLSAGQYFEITLSYDINKFYIDILDGP

SEQ ID NO: 19 - protein, 46 aa, chicken
VCNSMFQNNWGKEERTAPRFPFEPGTPFKLQVLCEGDHFKVAVNDA

SEQ ID NO: 20 - protein, 45 aa, rat
VFNTMQSGQWGKEEKKKSMPFQKGHHFELVFMVMSEHYKVVVNGT

SEQ ID NO: 21 - protein, 45 aa, mouse
VFKTMQSGQWGKEEKKKSMPFQKGKHFELVFMVMPEHYKVVVNGN

SEQ ID NO: 22 - protein, 46 aa, human
VCNSKDGGAWGTEQREAVFPFQPGSVAEVCITFDQANLTVKLPDGY

SEQ ID NO: 23 - protein, 45 aa, human
VFNTLQGGKWGSEERKRSMPFKKGAAFELVFIVMAEHYKVVVNGN

SEQ ID NO: 24 - protein, 34 aa, nematode
EFKDYEHRLPLSSISHLSIDGDLYLNHVHWGGKY

SEQ ID NO: 25 - protein, 29 aa, eel
NLEFPNRYSKEFLPFLSLAGDARLTLVKE

SEQ ID NO: 26 - protein, 34 aa, chicken
HLLQFNFREKKLNGITKLCIAGDITLTSVLTSMI

SEQ ID NO: 27 - protein, 47 aa, rat
PFYEYGHRLPLQMVTHLQVDGDLELQSINFLGGQPAASQYPGTMTIP

SEQ ID NO: 28 - protein, 47 aa, mouse
SFYEYGHRLPVQMVTHLQVDGDLELQSINFLGGQPAAAPYAGAMTIP

SEQ ID NO: 29 - protein, 30 aa, human
EFKFPNRLNLEAINYMAADGDFKIKCVAFD

SEQ ID NO: 30 - protein, 45 aa, human
PFYEYGHRLPLQMVTHLQVDGDLQLQSINFIGGQPLRPQGPPMMP

SEQ ID NO: 31 - protein, 23 aa, nematode
YPVPYESGLANGLPVGKSLLVFG

SEQ ID NO: 32 - protein, 47 aa, rat
AYPSAGYNPQMNSLPVMAGPPIFNPPVPYVGTLQGGLTARRTIIIKG

SEQ ID NO: 33 - protein, 50 aa, mouse
AYPAGSPGYNPPQMNTLPVMTGPPVFNPRVPYVGALQGGLTLPRTIIIKG

SEQ ID NO: 34 - protein, 47 aa, human
PYPGPGHCHQQLNSLPTMEGPPTFNPVPYFGRLQGGLTARRTIIIKG

SEQ ID NO: 35 - protein, 49 aa, nematode
TVEKKAKRFHVNLLRKNGDISFHFNPRFDEKHVIRNSLAANEWGNEERE

SEQ ID NO: 36 - protein, 49 aa, rat
YVLPTAKNLIINFKVGSTGDIAFHMNPRIGDCVVRNSYMNGSWGSEERK

SEQ ID NO: 37 - protein, 49 aa, mouse
YVLPTARNFVINFKVGSSGDIALHLNPRIGDSVVRNSFMNGSWGAEERK

SEQ ID NO: 38 - protein, 49 aa, human
YVPPTGKSFAINFKVGSSGDIALHINPHGNGTVVRNSLLNGSWGSEEKK

SEQ ID NO: 39 - protein, 48 aa, nematode
IPYNPFGAGQFFDLSIRCGTDRFKVFANGQHLFDFSHRFQAFQRVDML

SEQ ID NO: 40 - protein, 48 aa, rat
VAYNPFGPGQFFDLSIRCGMDRFKVFANGQHLFDFSHRFQAFQMVDTL

SEQ ID NO: 41 - protein, 48 aa, mouse
TTHNPFGPGQFFDLSIRCGLDRFKVYANGQHLFDFAHPSRAFQRVDTL

SEQ ID NO: 42 - protein, 48 aa, human; identical to SEQ ID NO: 41

SEQ ID NO: 43 - protein, 14 aa, nematode
QISGDIELSGIQIQ

SEQ ID NO: 44 - protein, 13 aa, rat
EIKGDITLSYVQI

SEQ ID NO: 45 - protein, 8 aa, mouse
EINGDITL

SEQ ID NO: 46 - protein, 13 aa, human
EIQGDVTLSYVQI

SEQ ID NO: 47 - protein, 914 aa, human
CCAGCCAACGAGCGGAAAATGGCAGACAATTTTTCGCTCCATGATGCGTTATCTGGGTCTGGAAACCCAAACCCTCAAGGATGGCCTGGCGCATGGGGGAACCAGCCTGCTGGGGCAGGGGGCTACCCAGGGGCTTCCTATCCTGGGGCCTACCCCGGGCAGGCACCCCCAGGGGCTTATCCTGGACAGGCACCTCCAGGCGCCTACCATGGAGCACCTGGAGCTTATCCCGGAGCACCTGCACCTGGAGTCTACCCAGGGCCACCCAGCGGCCCTGGGGCCTACCCATCTTCTGGACAGCCAAGTGCCCCCGGAGCCTACCCTGCCACTGGCCCCTATGGCGCCCCTGCTGGGCCACTGATTGTGCCTTATAACCTGCCTTTGCCTGGGGGAGTGGTGCCTCGCATGCTGATAACAATTCTGGGCACGGTGAAGCCCAATGCAAACAGAATTGCTTTAGATTTCCAAAGAGGGAATGATGTTGCCTTCCACTTTAACCCACGCTTCAATGAGAACAACAGGAGAGTCATTGTTTGCAATACAAAGCTGGATAATAACTGGGGAAGGGAAGAAAGACAGTCGGTTTTCCCATTTGAAAGTGGGAAACCATTCAAAATACAAGTACTGGTTGAACCTGACCACTTCAAGGTTGCAGTGAATGATGCTCACTTGTTGCAGTACAATCATCGGGTTAAAAAACTCAATGAAATCAGCAAACTGGGAATTTCTGGTGACATAGACCTCACCAGTGCTTCATATACCATGATATAATCTGAAAGGGGCAGATTAAAAAAAAAAAAAAAGAATCTAAACCTTACATGTGTAAAGGTTTCATGTTCACTGTGAGTGAAAATTTTTACATTCATCAATATCCCTCTTGTAAGTCATCTACTTAATAAATATTACAGTGAAAG